(19) United States
(12) Patent Application Publication
(10) Pub. No.: US 2026/0355531 A1
(43) Pub. Date: Nov. 12, 2026

(54) WEARABLE EAR TREATMENT APPARATUS AND ASSOCIATED SYSTEM AND METHOD

(71) Applicant: Buckeye Investments LLC, Hyde Park, UT (US)

(72) Inventors: Sam Adams, Bluffdale, UT (US); Jim Blotter, Hyde Park, UT (US); Nathan Erickson, Beaverdam, UT (US); Micah Parrish, North Logan, UT (US)

(21) Appl. No.: 19/669,917

(22) Filed: May 6, 2026

Related U.S. Application Data
(60) Provisional application No. 63/801,120, filed on May 6, 2025.

(51) Int. Cl. A61N 5/06

(52) U.S. Cl. CPC A61N 5/0603; A61N 5/0616; A61N 2005/0605; A61N 2005/063; A61N 2005/0647; A61N 2005/0651; A61N 2005/0661

(57) ABSTRACT

An ear treatment device includes a light disperser coupled with the light source in light receiving communication with the light source and a light guide sized to fit within an outer ear canal of an ear and coupled with the light disperser in light receiving communication with the light disperser. The light guide includes an elongate member made of a UVB-transmissive material, including a proximal end, fixed to the light disperser, and including a distal end, spaced away from the light disperser. The light guide also includes an optical stop fixed to the distal end of the elongate member and configured to substantially inhibit the transmission of the UVB light longitudinally beyond the distal end of the elongate member.

### CROSS-REFERENCE TO RELATED APPLICATION

This application claims the benefit of U.S. Provisional Patent Application No. 63/801,120, filed May 6, 2025, which is incorporated herein by reference.

### FIELD

The present application relates generally to medical devices for treating skin conditions, and more specifically to a wearable device for treating skin conditions of the ear canal.

### BACKGROUND

Ear canal skin conditions, like eczema (e.g., atopic dermatitis), psoriasis, and otitis externa, can cause itching, redness, scaling, flaking and/or cracking of the inner wall of the outer ear canal and/or the conchal bowl just outside the ear canal. Such skin conditions can be triggered by allergies, irritants, or underlying skin conditions, among other causes.

Treatment of such conditions can be difficult because of the size and accessibility constraints of the outer ear canal. Additionally, effective treatment of ear canal skin conditions is limited partially due to such conditions falling between, and often overlooked by, two medical specialties: ear-nose-throat and dermatology.

### SUMMARY

The subject matter of the present application has been developed in response to the present state of the art, and in particular, in response to the problems and needs associated with treatment of skin conditions, associated with the outer ear canal and conchal bowl, that have not yet been fully solved by currently available techniques.

The following is a non-exhaustive list of examples, which may or may not be claimed, of the subject matter, disclosed herein.

Disclosed herein is an ear treatment device. The ear treatment device includes a light disperser coupled with the light source in light receiving communication with the light source and a light guide sized to fit within an outer ear canal of an ear and coupled with the light disperser in light receiving communication with the light disperser. The light guide includes an elongate member made of a UVB-transmissive material, including a proximal end, fixed to the light disperser, and including a distal end, spaced away from the light disperser. Additionally, the light guide includes an optical stop fixed to the distal end of the elongate member and configured to substantially inhibit the transmission of the UVB light longitudinally beyond the distal end of the elongate member. The preceding subject matter of this paragraph characterizes example 1 of the present disclosure.

The light disperser is made of the UVB-transmissive material. The preceding subject matter of this paragraph characterizes example 2 of the present disclosure, wherein example 2 also includes the subject matter according to example 1, above.

The optical stop includes a reflective surface within the elongate member. The reflective surface is configured to reflect the light in the elongate member radially outwardly away from the elongate member. The preceding subject matter of this paragraph characterizes example 3 of the present disclosure, wherein example 3 also includes the subject matter according to any of examples 1-2, above.

The optical stop includes a blunt end outside the elongate member and opposite the reflective surface. The preceding subject matter of this paragraph characterizes example 4 of the present disclosure, wherein example 4 also includes the subject matter according to example 3, above.

The optical stop includes a metallic coating. The metallic coating defines the reflective surface. The preceding subject matter of this paragraph characterizes example 5 of the present disclosure, wherein example 5 also includes the subject matter according to any of examples 3-4, above.

The UVB light has a wavelength between, and inclusive of, 300 nanometers (nm) and 320 nm. The preceding subject matter of this paragraph characterizes example 6 of the present disclosure, wherein example 6 also includes the subject matter according to any of examples 1-5, above.

The ear treatment device further includes a light emitter including the light source and an earpiece including the light disperser and the light guide. The light emitter is selectively attachable to and detachable from the earpiece. The preceding subject matter of this paragraph characterizes example 7 of the present disclosure, wherein example 7 also includes the subject matter according to any of examples 1-6, above.

The light emitter includes at least one first magnetic coupling element. The earpiece includes at least one second magnetic coupling element. The light emitter and the earpiece are selectively attachable via magnetic coupling between the at least one first magnetic coupling element of the light emitter and the at least one second magnetic coupling element of the earpiece. The preceding subject matter of this paragraph characterizes example 8 of the present disclosure, wherein example 8 also includes the subject matter according to example 7, above.

The ear treatment device further includes a housing surrounding and at least partially enclosing the light disperser. The housing is made of a second UVB-blocking material. The preceding subject matter of this paragraph characterizes example 9 of the present disclosure, wherein example 9 also includes the subject matter according to any of examples 1-8, above.

The light disperser has an outer peripheral shape that is non-circular and asymmetrical. The light guide is off-center relative to an outer periphery of the light disperser. The preceding subject matter of this paragraph characterizes example 10 of the present disclosure, wherein example 10 also includes the subject matter according to any of examples 1-9, above.

Further disclosed herein is an ear treatment system. The ear treatment system includes two light sources each selectively and independently operable to generate ultraviolet B (UVB) light. The ear treatment system also includes two light applicators each including a light disperser coupled with a corresponding one of the light sources in light receiving communication with the corresponding one of the light sources and a light guide sized to fit within an outer ear canal of an ear and coupled with the light disperser in light receiving communication with the light disperser. The light guide includes an elongate member made of a UVB-transmissive material, including a proximal end, fixed to the light disperser, and including a distal end, spaced away from the light disperser. The light guide also includes an optical stop fixed to the distal end of the elongate member and configured to substantially inhibit the transmission of the UVB light longitudinally beyond the distal end of the elongate member. The ear treatment system further includes a control device communicatively connected with the two light sources via one of wired connection or a wireless connection and configured to independently control operation of each one of the light sources. The preceding subject matter of this paragraph characterizes example 11 of the present disclosure.

The ear treatment system further includes two light emitters each including a corresponding one of the two light sources and two earpieces each including a corresponding one of the two light applicators. Each one of the two light emitters is selectively attachable to and detachable from a corresponding one of the two earpieces. The preceding subject matter of this paragraph characterizes example 12 of the present disclosure, wherein example 12 also includes the subject matter according to example 11, above.

The ear treatment system further includes a test attachment including a second light applicator. Each one of the two light emitters is separately selectively attachable to and detachable from the test attachment. The preceding subject matter of this paragraph characterizes example 13 of the present disclosure, wherein example 13 also includes the subject matter according to example 12, above.

The ear treatment system further includes two light emitters each including a corresponding one of the light sources and two earpieces each including a corresponding one of the two light applicators. Each one of the two light emitters includes at least one first magnetic coupling element. Each one of the two earpieces includes at least one second magnetic coupling element. The test attachment includes at least one third magnetic coupling element. Each one of the two light emitters and a corresponding one of the two earpieces are selectively attachable via magnetic coupling between the at least one first magnetic coupling element and the at least one second magnetic coupling element. The test attachment and each one of the two light emitters are separately selectively attachable via magnetic coupling between the at least one first magnetic coupling element and the at least one third magnetic coupling element. The preceding subject matter of this paragraph characterizes example 14 of the present disclosure, wherein example 14 also includes the subject matter according to example 13, above.

The ear treatment system further includes two light emitters each includes a corresponding one of the light sources and two earpieces each includes a corresponding one of the two light applicators and a first housing to which the corresponding one of the two light applicators is fixed. The test attachment further includes a second housing to which the second light applicator is fixed. For each one of the two light applicators, the light disperser and the light guide extend from and are not surrounded by the first housing. The second light applicator is surrounded by the second housing. The preceding subject matter of this paragraph characterizes example 15 of the present disclosure, wherein example 15 also includes the subject matter according to any of examples 13-14, above.

For each one of the two light applicators, the light disperser continuously converges in a direction away from the first housing and at least a portion of the light guide has a constant diameter along a length of the light guide. The second light applicator includes a flat surface that is flush with a distal-most portion of the second housing. The preceding subject matter of this paragraph characterizes example 16 of the present disclosure, wherein example 16 also includes the subject matter according to example 15, above.

The control device is configured to operate a corresponding one of the two light emitters in a test mode when the corresponding one of the two light emitters is selectively attached to the test attachment. The control device is configured to operate the two light emitters in a treatment mode when each one of the two light emitters is selectively attached to the corresponding one of the two earpieces. The preceding subject matter of this paragraph characterizes example 17 of the present disclosure, wherein example 17 also includes the subject matter according to any of examples 13-16, above.

In the test mode, the control device incrementally increases the exposure of the light generated by the light source of the corresponding one of the two light emitters to determine a threshold exposure of light associated with a detectable skin response. In the treatment mode, the control device controls an intensity of the light generated by the light sources of the two light emitters based on the threshold exposure. The preceding subject matter of this paragraph characterizes example 18 of the present disclosure, wherein example 18 also includes the subject matter according to example 17, above.

Additionally disclosed herein is a method of treating a skin condition of an outer ear canal. The method includes inserting a light guide of an ear treatment device into the outer ear canal of an ear of a patient. The method also includes generating light, having a wavelength between, and inclusive of, 300 nanometers (nm) and 320 nm. The method further includes passing the light through an elongate member of the light guide in an axial direction along a central axis of the elongate member. The method additionally includes redirecting the light in the elongate member outwardly away from the central axis, out of the elongate member, and into contact with the skin condition of the outer ear canal. The preceding subject matter of this paragraph characterizes example 19 of the present disclosure.

The method further includes, prior to inserting the light guide of the ear treatment device into the outer ear canal of the ear of the patient, selectively attaching a test attachment to a light emitter to form a skin testing device, applying the skin testing device against a portion of skin of the patient away from the ear, incrementally increasing the exposure of the light until a detectable skin response occurs in the portion of the skin, identifying the exposure of the light at which the detectable skin response occurs as a threshold exposure, detaching the test attachment from the light emitter, and selectively attaching an earpiece to the light emitter. The earpiece includes the light guide and the earpiece and the light emitter form the ear treatment device. The preceding subject matter of this paragraph characterizes example 20 of the present disclosure, wherein example 20 also includes the subject matter according to example 19, above.

The described features, structures, advantages, and/or characteristics of the subject matter of the present disclosure may be combined in any suitable combination in one or more examples. In the following description, numerous specific details are provided to impart a thorough understanding of examples of the subject matter of the present disclosure. One skilled in the relevant art will recognize that the subject matter of the present disclosure may be practiced without one or more of the specific features, details, components, materials, and/or methods of a particular example. In other instances, additional features and advantages may be recognized in certain examples that may not be present in all examples or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

### BRIEF DESCRIPTION OF THE DRAWINGS

In order that the advantages of the subject matter may be more readily understood, a more particular description of the subject matter briefly described above will be rendered by reference to specific examples that are illustrated in the appended drawings. Understanding that these drawings, which are not necessarily drawn to scale, depict only certain examples of the subject matter and are therefore not to be considered limiting of its scope, the subject matter will be described and explained with additional specificity and detail through the use of the drawings, in which:

FIG. 1 is a side view of an ear treatment device, shown inserted into an outer ear canal of an ear, according to one or more examples of the present disclosure;

FIG. 2 is a side view of the ear treatment device of FIG. 1, showing light generated by a light source of the ear treatment device relative to an outer ear canal, according to one or more examples of the present disclosure;

FIG. 3 is a side view of an ear treatment device, according to one or more examples of the present disclosure;

FIG. 4 is a cross-sectional side view of a cup assembly of the ear treatment device of FIG. 3, shown in a position over an ear, according to one or more examples of the present disclosure;

FIG. 5 is a schematic view of an ear treatment system, according to one or more examples of the present disclosure;

FIG. 6 is a schematic view of another ear treatment system, according to one or more examples of the present disclosure;

FIG. 7 is a perspective view of a light emitter of an ear treatment device, according to one or more examples of the present disclosure;

FIG. 8 is a perspective view of an earpiece of an ear treatment device, according to one or more examples of the present disclosure;

FIG. 9 is a front view of the earpiece of FIG. 8, according to one or more examples of the present disclosure;

FIG. 10 is an alternative perspective view of the earpiece of FIG. 8, according to one or more examples of the present disclosure;

FIG. 11 is an exploded perspective view of the earpiece of FIG. 8, according to one or more examples of the present disclosure;

FIG. 12 is an exploded side view of an ear treatment device that includes the light emitter of FIG. 7 and the earpiece of FIG. 8, according to one or more examples of the present disclosure;

FIG. 13 is a side view of the ear treatment device of FIG. 12, according to one or more examples of the present disclosure;

FIG. 14 is a side view of the ear treatment device of FIGS. 12 and 13, showing light generated by a light source of the ear treatment device relative to an outer ear canal, according to one or more examples of the present disclosure;

FIG. 15 is a perspective view of a test attachment of an ear treatment system, according to one or more examples of the present disclosure;

FIG. 16 is another perspective view of the test attachment of FIG. 15, according to one or more examples of the present disclosure;

FIG. 17 is an exploded perspective view of the test attachment of FIGS. 15 and 16, according to one or more examples of the present disclosure;

FIG. 18 is an exploded side view of a skin testing device of an ear treatment system, according to one or more examples of the present disclosure;

FIG. 19 is a side view of the skin testing device of FIG. 18, according to one or more examples of the present disclosure;

FIG. 20 is a side view of the skin testing device of FIGS. 18 and 19, showing light generated by a light source of the skin testing device relative to a portion of skin away from the ear, according to one or more examples of the present disclosure; and

FIG. 21 is a schematic flow diagram of a method of treating a skin condition of an outer ear canal, according to one or more examples of the present disclosure.

### DETAILED DESCRIPTION

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

Disclosed herein is an ear treatment device, and associated system and method of use, that helps overcome at least some of the shortcomings of currently available techniques for treating skin conditions of the outer ear canal. As used herein, the conchal bowl of the ear will be considered part of the outer ear canal unless otherwise noted. The ear treatment device is wearable by a user at least partially over and into the outer ear canal. When worn, the ear treatment device is selectively operable to deliver light (i.e., electromagnetic radiation) to the surface of the outer ear canal. The wavelength, intensity, and/or duration of the light is particularly tailored to treat skin conditions of the outer ear canal. The ear treatment device is designed to redirect and concentrate the delivery of the light to the surface of the outer ear canal and to restrict or prevent light from entering the inner ear canal. The ergonomic, lightweight, and wearable nature of the ear treatment device enables a user to easily and comfortably receive treatment in a medical treatment facility or at home. In some examples, a user can perform day-to-day activities while the ear treatment device is worn by the user and is treating a skin condition of the outer ear canal.

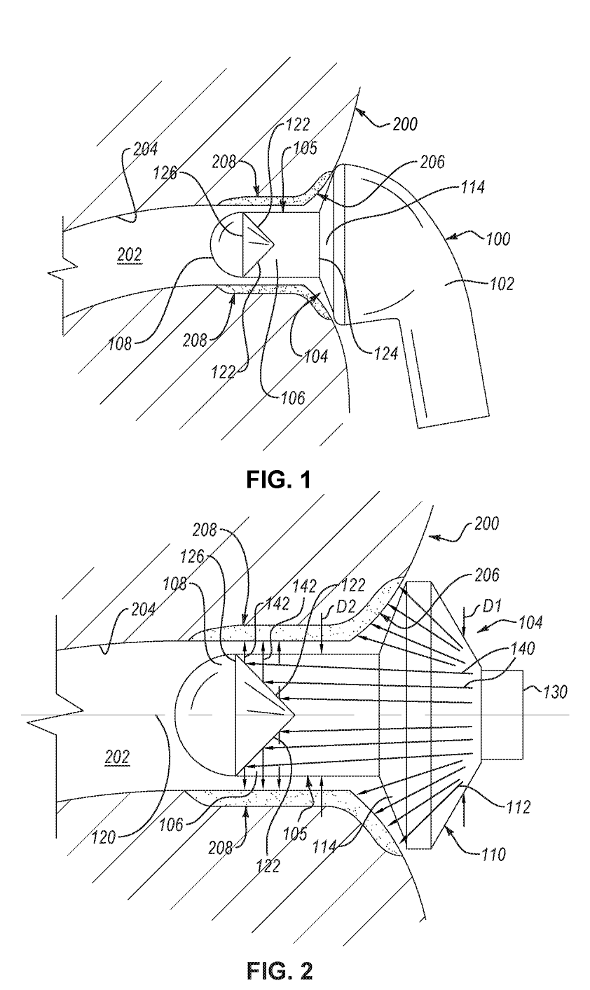

Referring to FIG. 1, according to one example, an ear treatment device 100 is shown. The ear treatment device 100 of FIG. 1 resembles an earbud and is worn by a user in a manner similar to an earbud. The ear treatment device 100 includes a housing 102 and a light applicator 104 coupled to and at least partially housed within the housing 102. Generally, the housing 102 is located externally to the ear when worn by a user and the light applicator 104 is partially located within the outer ear canal 202 of an ear 200 of the user when worn. More specifically, the ear treatment device 100 is configured so that when properly worn by a user, no portion of the housing 102 is located within the outer ear canal 202 of the user and a light guide 105 of the light applicator 104 is located mostly, if not entirely, within the outer ear canal 202. The light guide 105 includes an elongate member 106 and an optical stop 108 fixed to (e.g., adhered to, formed in, coupled to, etc.) a distal end 126 of the elongate member 106, which is opposite a proximal end 124 of the elongate member 106. When worn by a user, the light guide 105 is inserted into the outer ear canal 202 so that the distal end 126 of the elongate member 106 (otherwise known as the cantilevered end of the elongate member 106) is located further inside the outer ear canal 202 than the proximal end 124. The distance the light guide 105 extends into the outer ear canal 202 can vary based on the length of the elongate member 106. Accordingly, the optical stop 108, being fixed to the distal end 126, is also located further inside the outer ear canal 202 than the proximal end 124 of the elongate member 106.

As used herein, the term “elongate member” refers to an elongate structure configured to transmit electromagnetic radiation, including ultraviolet (UV) light, along at least a portion of its length. The elongate member can be hollow (e.g., a tube) or solid (e.g., a cylinder). In the illustrated examples, the elongate member 106 is solid and the optical stop 108 is embedded at least partially within the material of the elongate member 106. The elongate member 106 can have any of various cross-sectional shapes, and the size of the cross-section may be constant or vary along its length (e.g., tapered or stepped). For example, in the illustrated implementations, the elongate member 106 has a circular cross-sectional shape with a constant diameter along the length. However, in other examples, the elongate member 106 has a non-circular cross-sectional shape, such as one of an oval, a polygon (e.g., triangle, square, rectangle, hexagon, and the like), or other shape. The cross-sectional shape of the elongate member 106 can be constant or along its length. Also, the cross-sectional shape of the elongate member 106 can be symmetrical or asymmetrical about one or more planes. In certain examples, the elongate member 106 is straight, as shown, but can be curved in other examples. Additionally, the elongate member 106 can be rigid, semi-rigid, or flexible.

As used herein, the term “optical stop” (also referred to as a “light-blocking element”, a “light-termination element”, or a “light barrier”) refers to a structure disposed at or near the distal end of the elongate member and configured to substantially inhibit the transmission of light in a longitudinal or axial direction beyond the distal end of the elongate member. The optical stop 108 can provide such functionality using one or more structural mechanisms, such as reflection, absorption, scattering, diffusion, refraction, or geometric redirection. As mentioned below, the optical stop 108 can be made of an opaque material (i.e., a material opaque to UVB transmissions (i.e., UVB-blocking material)), have reflective coatings or layers (which can also be considered a UVB-blocking material), include diffusive or scattering structures, a change in geometry, or combinations thereof.

As shown in FIGS. 1 and 2, in one example, the optical stop 108 includes a reflective surface 122 located within the elongate member 106. The optical stop 108 can be fixed to the distal end 126 of the elongate member 106 so that the reflective surface 122 extends from the distal end 126 toward the proximal end 124. The reflective surface 122 has a general conical shape with a pointed end. A central axis of the conical shape is coaxial with a central axis 120 of the elongate member 106. In the illustrated example, the reflective surface 122 defines a 45-degree right cone. However, in other examples, the reflective surface 122 can define right cones having angles different than 45-degrees. Moreover, although the reflective surface 122 in the illustrated examples is linear along a plane parallel to the central axis 120, in other examples, the reflective surface 122 can be concave or convex along a plane parallel to the central axis 120. The optical stop 108 also includes a blunt end opposite the pointed end of the reflective surface 122 and located outside of the elongate member 106. Moreover, the blunt end of the optical stop 108 extends distally away from the distal end 126 of the elongate member 106. In this manner, the blunt end of the optical stop 108 defines a leading end of the ear treatment device 100 as the ear treatment device 100 is inserted into the outer ear canal 202. Accordingly, the non-pointed shape of the blunt end facilitates smooth, low-resistance, and comfortable insertion of the light guide 105 into the outer ear canal 202. In the illustrated example, the blunt end has a semi-spherical shape, but can have other non-pointed shapes as desired.

Referring to FIG. 2, the light applicator 104 further includes a light disperser 110 and the ear treatment device 100 further includes a light source 130. The light source 130 is coupled to the light disperser 110 so that light generated by the light source 130 is transmitted into the light disperser 110. The light disperser 110 includes a diverging portion 112 and a converging portion 114 that define a corresponding one of a diverging cavity and a converging cavity within the light disperser 110. The diverging portion 112 and the diverging cavity diverge, or increase in size, in a direction away from the light source 130 and toward the light guide 105. In contrast, the converging portion 114 and the converging cavity converge, or decrease in size, in the direction away from the light source 130 and toward the light guide 105. Accordingly, in some examples, the light disperser 110 has a disk-like shape. The proximal end 124 of the elongate member 106 of the light guide 105 extends from (e.g., is attached to) the converging portion 114 of the light disperser 110.

The size of the light disperser 110 can be defined by a maximum dimension (e.g., diameter D1 when the light disperser 110 is circular) of the light disperser 110 and the size of the elongate member 106 of the light guide 105 can be defined by a maximum dimension (e.g., diameter D2 when the elongate member 106 is circular) of the elongate member 106. The diameter D1 of the light disperser 110 increases along the diverging portion 112 and decreases along the converging portion 114, in the direction away from the light source 130. In contrast, in the illustrated examples, the diameter D2 of the elongate member 106 is constant in the direction away from the light source 130. Moreover, in some examples, the minimum value of the diameter D1 of the converging portion 114 is equal to the diameter D2 of the elongate member 106.

The light source 130 is operable to generate light at a wavelength, intensity, and duration conducive to treating a skin condition 208 on the surface 204 of the outer ear canal 202. In some examples, the light source 130 is controllable to adjust the wavelength and intensity of the light it generates. For example, the light source 130 can be controlled to generate light at one of a plurality of wavelengths and/or intensities based on the type or intensity of the skin condition 208. However, in other examples, the wavelength and intensity of the light generated by the light source 130, although selected to treat the skin condition 208, is fixed. In preferred examples, the light source 130 is configured to generate ultraviolet light or light having a wavelength within the ultraviolet spectrum. More specifically, in certain examples, the light source 130 generates narrowband mid-wave ultraviolet light (UVB), which includes light having a wavelength between, and inclusive of, 300 nanometers (nm) and 320 nm generally, and between, and inclusive of, 305 nm and 315 nm, such as 308 nm, more specifically. The light source 130 can include one or more light-emitting diodes (LEDs) configured to generate ultraviolet light. Alternatively, or additionally, the light source 130 can include one or more ultraviolet lightbulbs. The light generated by the light source 130 is non-coherent or multi-directional light, in some examples, such that the beam of light generated by the light source 130 diverges after being generated.

The ear treatment device 100 is made of different materials based on weight and light transmissibility. Generally, the ear treatment device 100 is made of lightweight materials to promote comfort when wearing the ear treatment device 100. For example, the housing 102 and the light applicator 104 can be made of a polymeric material. However, the polymeric material of the housing 102 and the optical stop 108 of the light applicator 104 have a lower UVB light transmissibility than the polymeric material of the light disperser 110 and the elongate member 106 of the light guide 105. According to one example, the housing 102 and the optical stop 108 are made of a generally opaque plastic material, such as acrylonitrile butadiene styrene (ABS) plastic, that prevents the transmission of UVB light. In contrast, according to one example, the light disperser 110 and the elongate member 106 are made of an at least partially transparent material, such as an acrylic material, that is UVB transmissive (i.e., a UVB-transmissive material). More specifically, in certain examples, the light disperser 110 and the elongate member 106 are made of materials capable of transmitting, guiding, diffusing, scattering, or otherwise directing UVB light, including through total internal reflection, surface reflection, refraction, or bulk transmission. In some examples, the light disperser 110 and/or the elongate member 106 may include coatings, claddings, surface treatments, or internal structures configured to enhance transmission, control dispersion, or direct UVB light outward (e.g., radially outward) toward surrounding tissue, including the outer ear canal 202.

To promote reflectively of the reflective surface 122 of the optical stop 108, in certain examples, the optical stop 108 includes a reflective coating that defines the reflective surface 122. In other words, a reflective coating, made of a reflective material, such as a metallic material (e.g., aluminum), can be coated on or applied to a plastic conical portion of the optical stop 108.

Referring again to FIG. 2, a method of using the ear treatment device 100 to treat a skin condition 208 of the outer ear canal 202 (including the conchal bowl 206) includes coupling the ear treatment device 100 with the ear 200 of a user by inserting the light guide 105 into the outer ear canal 202. In some examples, coupling the ear treatment device 100 with the ear 200 also includes abutting the converging portion 114 of the light disperser 110 against a portion of the conchal bowl 206 or rim of the outer ear canal 202. The size of the light guide 105 (e.g., the diameter D2 of the elongate member 106) is such that the elongate member 106 is self-retained within the outer ear canal 202. Retention of the elongate member 106 within the outer canal 202 can be provided by a friction fit between the elongate member 106 and the outer ear canal 202. Although not shown, in some examples, the ear treatment device 100 can include straps, wires, or other retention features that help keep the ear treatment device 100 coupled to the ear 200. When the light guide 105 is inserted into the outer ear canal, the outer surface of the elongate member 106 is substantially parallel to the surface of the outer ear canal 202 such that the outer surface of the elongate member 106 faces the surface of the outer ear canal 202. Moreover, when the light guide 105 is inserted into the outer ear canal 202, an external angled surface of the converging portion 114 faces the conchal bowl 206 or rim of the outer ear canal 202.

With the light guide 105 inserted into the outer ear canal 202, the light source 130 can be activated to generate incident light 140 at a desired wavelength, intensity, and duration. The incident light 140 generated by the light source 130 is shown as solid directional arrows in FIG. 2. The incident light 140, being non-coherent, radiates outwardly from the light source 130 in all directions. The incident light 140 initially passes through the at least partially translucent material of the light disperser 110. Some of the incident light 140 passing through the light disperser 110 is absorbed by the housing 102. Other portions of the incident light 140 pass through the light disperser 110 and contact (e.g., are absorbed) by the conchal bowl 206 or rim of the outer ear canal 202. More specifically, some of the incident light 140, propagating at an angle relative to the central axis 120, passes through the angled surface of the converging portion 114 of the light disperser 110 and impacts the conchal bowl 206 or rim of the outer ear canal 202. Yet other portions of the incident light 140, propagating generally parallel to the central axis 120, pass through the light disperser 110 and enter the elongate member 106 of the light guide 105. The incident light 140 propagates in a direction generally parallel to the central axis 120 as it travels through the elongate member 106.

Eventually, the incident light 140 propagating through the elongate member 106 impacts and is reflected off the reflective surface 122 as reflected light 142. Due to the angle of the reflective surface 122 relative to the central axis 120 of the elongate member 106, the reflected light 142 propagates in a direction that is angled relative to the central axis 120. In the illustrated example, the reflected light 142 propagates in a direction perpendicular relative to the central axis 120, and thus substantially perpendicular to the surface 204 of the outer ear canal 202. The direction perpendicular to the central axis 120 is considered a radial direction because it extends radially outwardly away from the central axis 120, rather than parallel to the central axis 120, which would be considered an axial direction. Because the elongate member 106 is at least partially translucent, the reflected light 142 passes through the wall of the elongate member 106 and impacts the surface 204 of the outer ear canal 202 head on or with a force vector that is perpendicular to the surface 204. Because the reflected light 142 impacts the surface 204 perpendicularly relative to the surface 204, the energy from the reflected light 142 is more efficiently transferred to the surface than if the reflected light 142 were at an oblique angle relative to the surface 204. This efficient transfer of energy from reflected light 142 to the surface 204 results in a more efficient treatment of the skin condition 208.

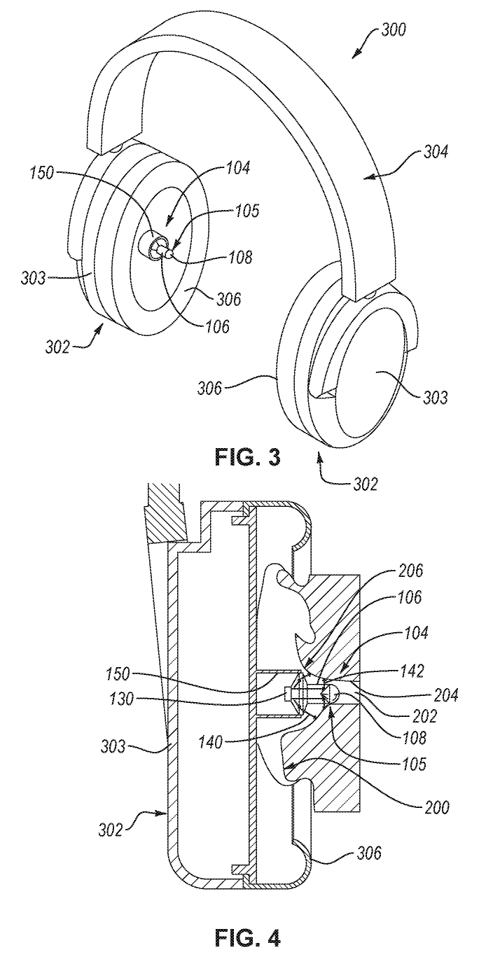

Referring to FIGS. 3 and 4, another example of an ear treatment device 300 is shown. The ear treatment device 300 resembles a headphone and is worn by a user in a manner similar to a headphone. The ear treatment device 300 includes two cup assemblies 302 and a headband 304. The headband 304 connects the cup assemblies 302 together and provides a support for keeping the ear treatment device 300 on the head of a user. Each one of the cup assemblies 302 includes an ear cup 303, an ear cushion 306, and a light applicator 104 surrounded by the ear cushion 306. The light applicator 104 is similar to the light applicator 104 of the ear treatment device 100. However, the light applicator 104 of the ear treatment device 300 includes a stand-off 150 that properly locates the light guide 105 for insertion into the outer ear canal 202 when the ear cup 303 and ear cushion 306 are placed over the ear 200. When inserted in the outer ear canal 202, the light applicator 104 is operable in the same manner as the light applicator 104 of the ear treatment device 100 to treat a skin condition of the outer ear canal 202.

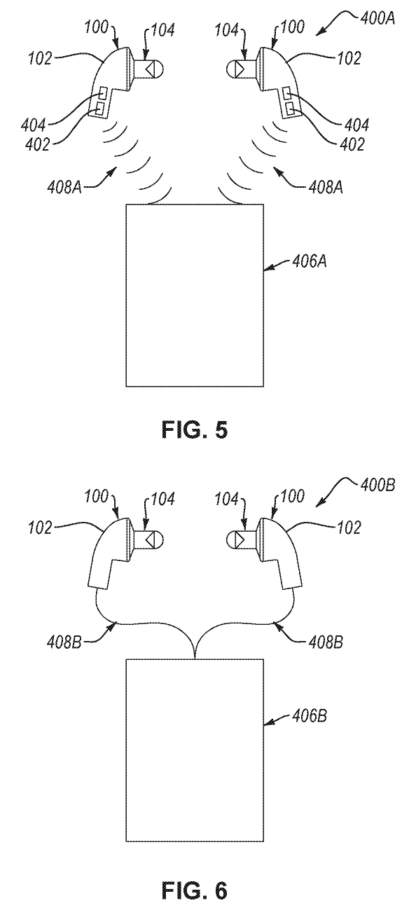

Referring to FIG. 5, one example of an ear treatment system 400A is shown. The ear treatment system 400A includes two ear treatment devices 100 wireless connected to a control device 406A. Each one of the ear treatment devices 100 includes a wireless transceiver 402 capable of receiving and transmitting wireless signals 408A (e.g., Wi-Fi signals, Bluetooth signals, and the like). Additionally, each one of the ear treatment devices 100 includes a controller 404 configured to control operation of the light applicator 104 in response to operational commands provided by the control device 406A via the wireless signals 408A. The control device 406A can be any of various control devices, such as smartphones, laptops, tablets, dedicated handheld devices, and the like.

Referring to FIG. 6, another example of an ear treatment system 400B is shown. The ear treatment system 400B is similar to the ear treatment system 400A of FIG. 5. However, instead of a wireless connection, the ear treatment devices 100 communicate with a control device 406B via a wired connection 408B. Accordingly, the ear treatment devices 100 of the ear treatment system 400B do not have wireless transceiver 402, in some examples, and do not have on-board controllers 404. Instead, in some examples, the controller is integrated into the control device 406B, which, in preferred examples, is a dedicated handheld device.

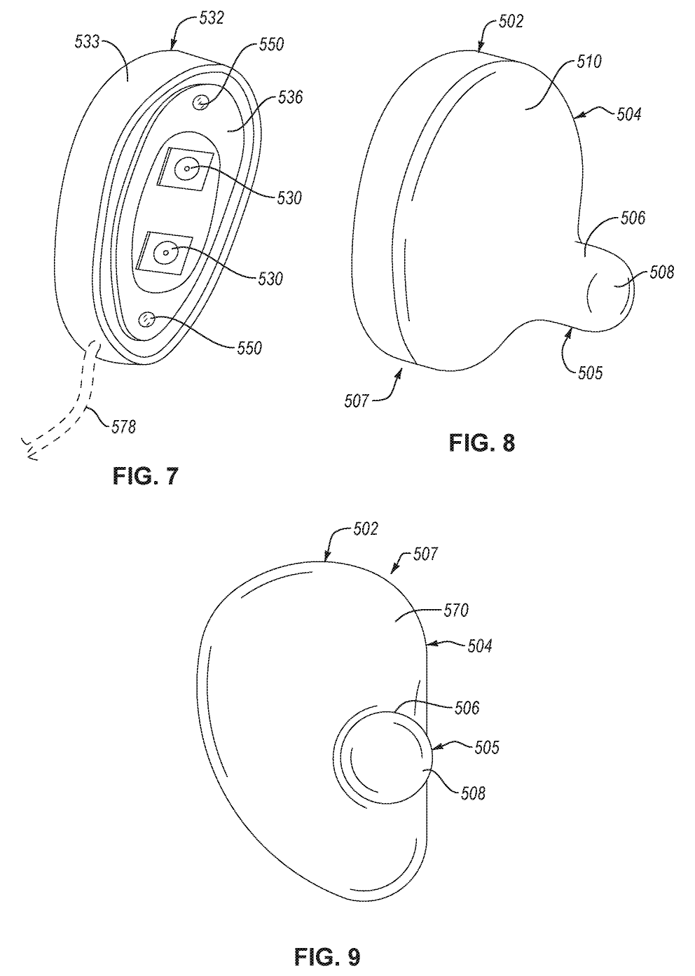

Referring to FIGS. 7-14, another example of an ear treatment device 500 is shown. The ear treatment device 500 is analogous to the ear treatment device 100 in some respects and shares some common features and functionality with the ear treatment device 100. For example, like the ear treatment device 100, the ear treatment device 500 is configured to be wearable by a user at least partially in the outer ear canal 202. Additionally, when worn, the ear treatment device 500 is selectively operable to deliver light to the surface 204 of the outer ear canal 202 to treat skin conditions 208 of the outer ear canal 202. However, the ear treatment device 500 is designed and includes alternative features that promote ergonomic design, light delivery efficiency, and versatility.

As shown in FIG. 7, the ear treatment device 500 includes a light emitter 532. The light emitter 532 includes a base 533 that retains light sources 530 each similar to the light source 130. More specifically, each one of the light sources 530 is operable to generate light at a wavelength, intensity, and duration conducive to treating a skin condition 208 on the surface 204 of the outer ear canal 202. In the illustrated example, the light emitter 532 includes two light sources 530 fixed to the base 533 and spaced apart from each other. Moreover, the light sources 530 are vertically and horizontally offset from each other to promote a broader and more uniform emission of light. The light sources 530 can have the same orientation (e.g., face the same direction), such that the light sources 530 generate and transmit light in the same general direction. However, in other examples, the light sources 530 have different orientations (e.g., face different directions or are angled relative to each other), such that the light sources 530 generate and transmit light in different directions, which can help to transmit light more broadly or help concentrate the light.

The base 533 can be made of a non-electrically conductive material, such as a polymeric material, and include receptacles for retaining the light sources 530. In some examples, the light emitter 532 includes a clear cover over the light sources 530 to protect the light sources 530 from impact damage while enabling the transmission of light. Additionally, the light emitter 532 includes electronic circuitry (not shown) configured to operate the light sources 530 in response to control commands received from a control device, such as the control devices 406A, 406B. The electronic circuitry can include wireless transceivers to receive control commands wirelessly or electronics that enable the receipt of control commands via a wired interface, such as wired connection 578. The base 533 can house a rechargeable battery for supplying electrical power to the light sources 530 or electrical power can be supplied to the light emitter 532 via the wired interface.

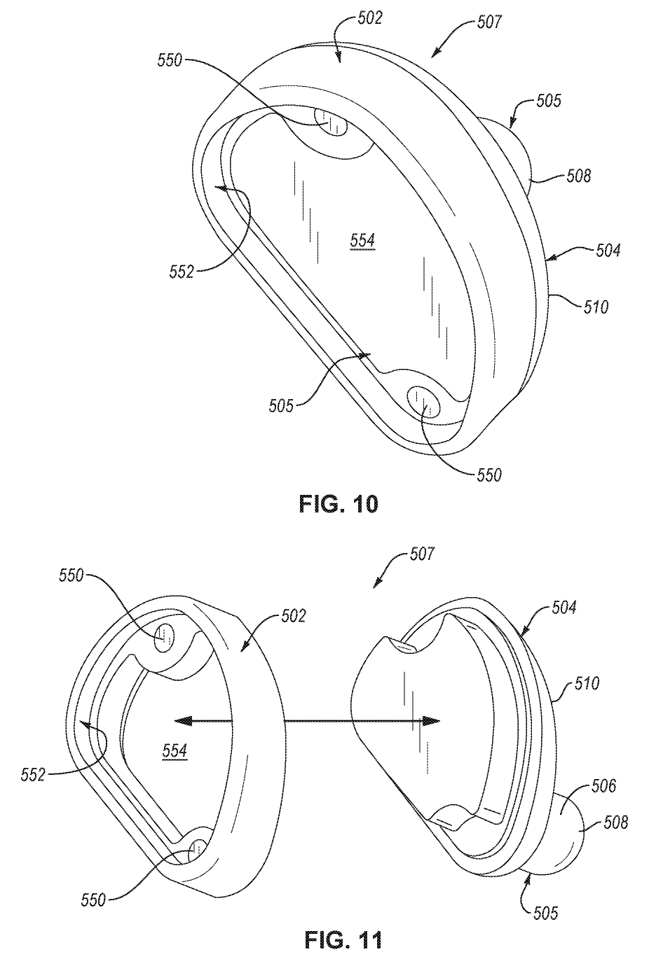

The light emitter 532 is configured to be selectively attachable to and detachable from an earpiece of the ear treatment device 500, such as the earpiece 507 shown in FIG. 8. To promote attachment to and detachment from the earpiece 507, the light emitter 532 includes magnetic coupling elements 550 (e.g., magnets or ferromagnetic material) retained in (e.g., press-fit into) the base 533 and the base 533 includes a mating feature 536. The mating feature 536 can be any of various types of features that promote alignment, orientation, and mating with the earpiece 507. In the illustrated example, the mating feature 536 is a boss, projection, or raised portion configured to be inserted into (e.g., nestably engaged with) a receptacle 552 of the earpiece (see, e.g., FIG. 10). However, the configuration can be reversed, such that the mating feature 536 is a recessed feature, receptacle, or cavity, into which a mating feature of the earpiece can be inserted. In one example, as shown, the magnets 550 are retained in the mating feature 536 of the base 533. The magnetic coupling elements 550 are positioned on the base 533 to correspond with the position of magnetic coupling elements 550 on the earpiece 507. More specifically, each one of the magnetic coupling elements 550 on the base 533 is positioned to magnetically engage a corresponding one of the magnetic coupling elements 550 of the earpiece 507. The magnetic force between magnetically engaged magnetic coupling elements promotes attachment of the light emitter 532 and the earpiece 507. As will be explained in more detail, the light emitter 532 can be detached from the earpiece 507 by pulling the light emitter 532 and the earpiece 507 apart from each other with a pull-apart force greater than the combined magnetic force of the coupled magnetic coupling elements. Although magnetic coupling elements are used in the illustrated examples to help couple together the light emitter 532 and the earpiece 507, in other examples, other techniques can be used, such as press-fitting, interference fitting, interlock fitting, and the like.

Referring to FIGS. 8-11, the earpiece 507 of the ear treatment device 500 includes a housing 502 and a light applicator 504 coupled to and at least partially housed within the housing 502. The housing 502 is made of a substantially opaque material such that light generated by the light emitter 532 does not pass through the housing 502. In the illustrated example, as shown in FIG. 11, the housing 502 has a ring-like shape or a continuous outer perimeter that defines a through-cavity 554. A proximal portion of the light applicator 504 is located within the through-cavity 554 and is fixed to the housing 502 via any one of various fixation techniques, such as adhesion, bonding, fastening, and the like. A distal portion of the light applicator 504 extends externally away from the housing 502 and is configured to be inserted into the outer ear canal 202 as will be explained below.

The magnetic coupling elements 550 (e.g., magnets or ferromagnetic material) of the earpiece 507 are coupled to or embedded within (e.g., press-fit into) the housing 502. Moreover, the magnetic coupling elements 550 are accessible within the receptacle 552, which is defined between the housing 502 and the light applicator 504 when coupled to the housing 502 (see, e.g., FIG. 10).

Like the light applicator 104, the light applicator 504 is made of an at least partially transparent material so that light generated by the light emitter 532 passes through the light applicator 504. The light applicator 504 includes a light disperser 510 and a light guide 505 seamlessly coupled to and extending from the light disperser 510. The light disperser 510 includes an outer peripheral shape that can match the peripheral outer shape of the housing 502. In some examples, the outer peripheral shape of the housing 502 and the light disperser 510 is non-circular and asymmetrical to correspond with the substantially non-circular and asymmetrical shape of the outer perimeter of the conchal bowl 206. The light disperser 510 converges in a direction away from the housing 502 to correspond with the converging nature of the conchal bowl 206 in a direction towards the outer ear canal 202. In other words, the outer surface of the light disperser 510 has a generally convex bulbous shape to promote at least partial nestability with the concave bowl shape of the conchal bowl 206. The light disperser 510 continuously converges toward a seamless interface with the light guide 505.

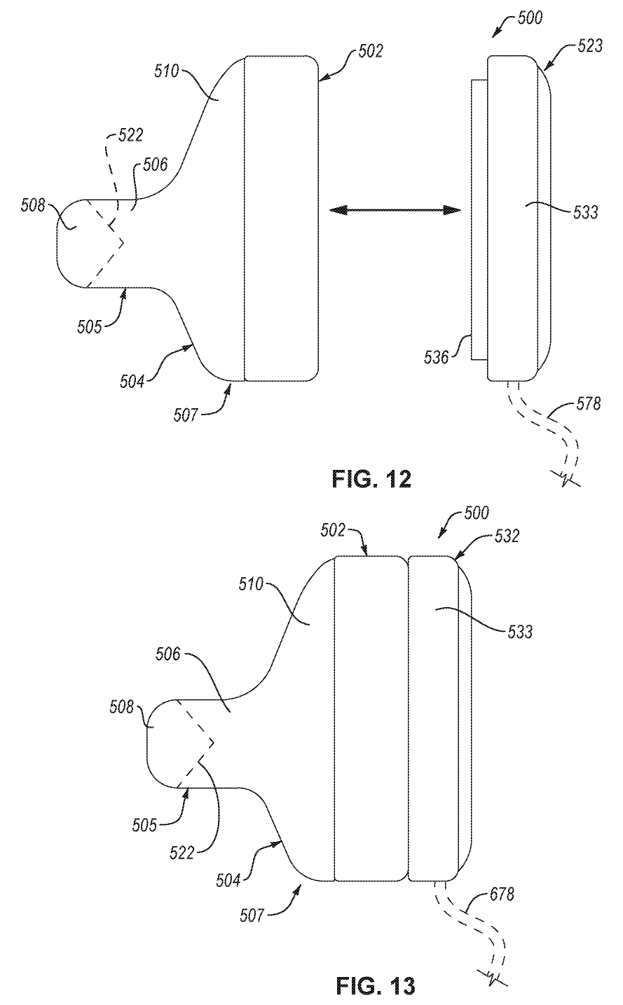

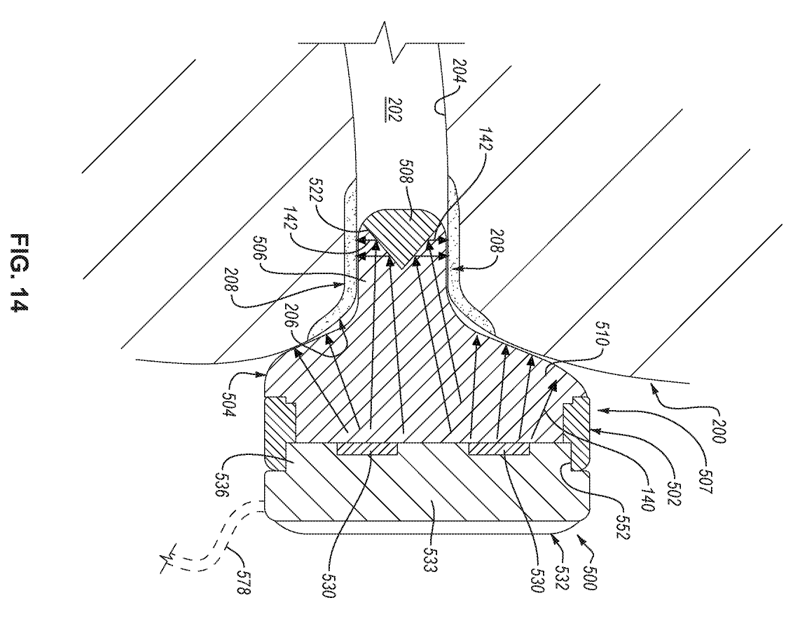

Referring to FIGS. 12 and 13, the light guide 505 has a generally circular cross-sectional shape and a generally constant diameter along a length of the light guide 505, similar to the light guide 105. The light guide 105 includes an elongate member 506 and an optical stop 508. Like the light guide 105, the elongate member 506 can be a solid structure and the optical stop 508 can be embedded into a distal end of the elongate member 506. A blunt end of the optical stop 508 defines a distal end of the light guide 505 and includes a reflective surface 522 located within the elongate member 506. The reflective surface 522 is configured to reflect light in the same way as the reflective surface 122 of the optical stop 108. Accordingly, the features and functionality described above with respect to the light guide 105 are applicable to the light guide 505.

As shown in FIGS. 8 and 9, the location of the light guide 505 is off-center relative to the outer perimeter or periphery of the light disperser 510 to better conform to the location of the outer ear canal 202 relative to the outer perimeter of the conchal bowl 206. In some examples, the light guide 505 is located at a lower-side portion of the lighter disperser 510.

To assemble the ear treatment device 500, the earpiece 507 and the light emitter 532 are brought together, as shown in FIG. 12, such that the mating feature 536 of the light emitter 532 is aligned with the receptacle 552 of the earpiece 507. Once aligned, the mating feature 536 can be inserted into the receptacle 552 until the magnetic coupling elements 550 of the light emitter 532 are magnetically coupled to corresponding magnetic coupling elements 550 of the earpiece 507, as shown in FIG. 13. The ear treatment device 500, when assembled, is ready to be inserted into the outer ear canal 202, as shown in FIG. 14. Similar to the ear treatment device 100, when the light guide 505 is positioned within the outer ear canal 202, the lighter disperser 510 abuts (e.g., nests with) at least a portion of the conchal bowl 206. The light guide 505 can be sized to engage the outer ear canal 202 with a friction fit, which helps to retain the ear treatment device 500 in engagement with the ear 200.

With the light guide 505 inserted into the outer ear canal 202, the light sources 530 can be activated to generate incident light 140 at a desired wavelength, intensity, and duration. The incident light 140 generated by the light sources 530 is shown as solid directional arrows in FIG. 14. The incident light 140, being non-coherent, radiates outwardly from the light sources 530 in all directions. The incident light 140 initially passes through the at least partially translucent material of the light disperser 510. Some of the incident light 140 passing through the light disperser 510 is absorbed by the housing 502. Other portions of the incident light 140 pass through the light disperser 510 and contact (e.g., are absorbed) by the conchal bowl 206. Yet other portions of the incident light 140 enter and propagate along the elongate member 506 of the light guide 505.

Eventually, the incident light 140 propagating through the elongate member 506 impacts and is reflected off the reflective surface 522 as reflected light 142. Due to the angle of the reflective surface 522 relative to the central axis 120 of the elongate member 106, the reflected light 142 propagates in a radial direction that is angled relative to a central axis of the outer ear canal 202. The reflected light 142 passes through the translucent material of the elongate member 106 and impacts the surface 204 of the outer ear canal 202 head on or with a force vector that is perpendicular to the surface 204.

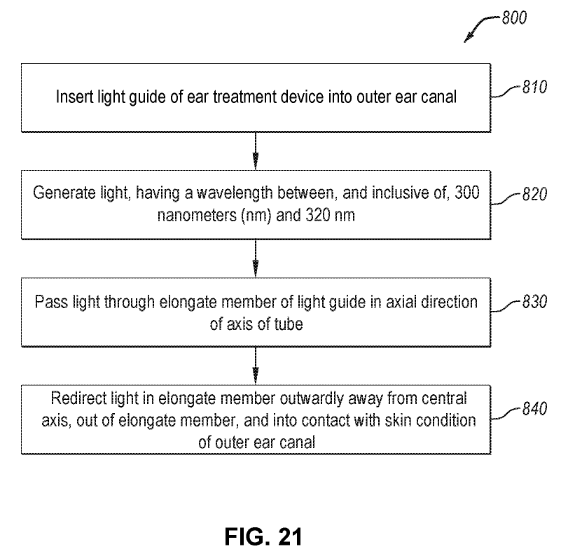

In view of the foregoing, one example of a method 800 of treating the skin condition 208 of the outer ear canal 202 is shown in FIG. 21. The method 800 includes (block 810) inserting the light guide 105 of the ear treatment device 100 into the outer ear canal 202 of the ear 200 of a patient. The method 800 additionally includes (block 820) generating light, having a wavelength between, and inclusive of, 300 nanometers (nm) and 320 nm. The method 800 also includes (block 830) passing the light through the elongate member 106 of the light guide 105 in an axial direction along the central axis 120 of the elongate member 106. The method 800 further includes (block 840) redirecting the light in the elongate member 106 radially outwardly away from the central axis 120, out of the elongate member 106, and into contact with the skin condition 208 of the outer ear canal 202. Although described in association with features of the ear treatment device 100, the method 800 can be practiced with other ear treatment devices, such as the ear treatment device 500 described below.

Prior to treating the skin condition 208 with the ear treatment device 500, in some examples, an understanding of the skin properties, such photosensitivity (e.g., sun sensitivity), of the patient can be helpful to determine the proper wavelength, intensity, and/or duration of the treatment. According to conventional techniques of treating various skin conditions, if needed, the photosensitivity of skin is determined by using a separate standalone device relative to the device used to treat the skin, which can add complexity and cost. Alternatively, the photosensitivity of skin can be determined in a separate setting by a separate device, which can lead to increased costs and delays.

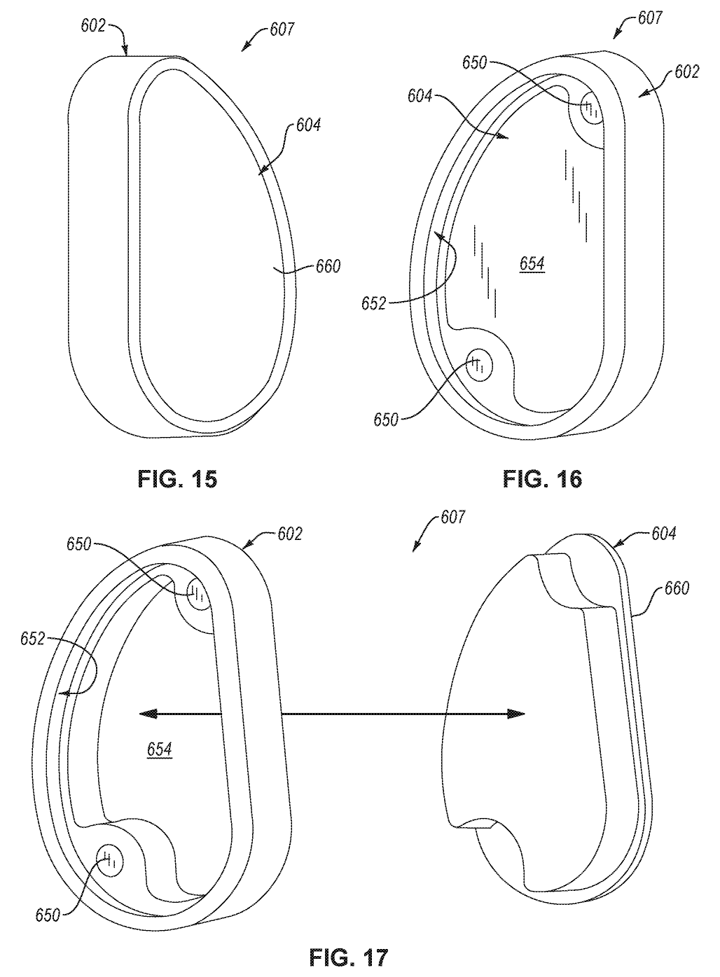

According to some examples of the present disclosure, the ear treatment device 500 forms part of an ear treatment system that includes the light emitter 532, the earpiece 507, and a test attachment 607 (see, e.g., FIGS. 15-17). The earpiece 507 and the test attachment 607 can be interchangeably coupled with the light emitter 532. In other words, the light emitter 532 can be interchangeably used to generate light for either the test attachment 607, for testing the photosensitivity of skin, or the earpiece 507, for treating skin conditions of the outer ear canal. Accordingly, the same system, in the same setting, can be used to test skin properties and treat skin conditions of the ear, which saves time, reduces cost, and promotes simplicity.

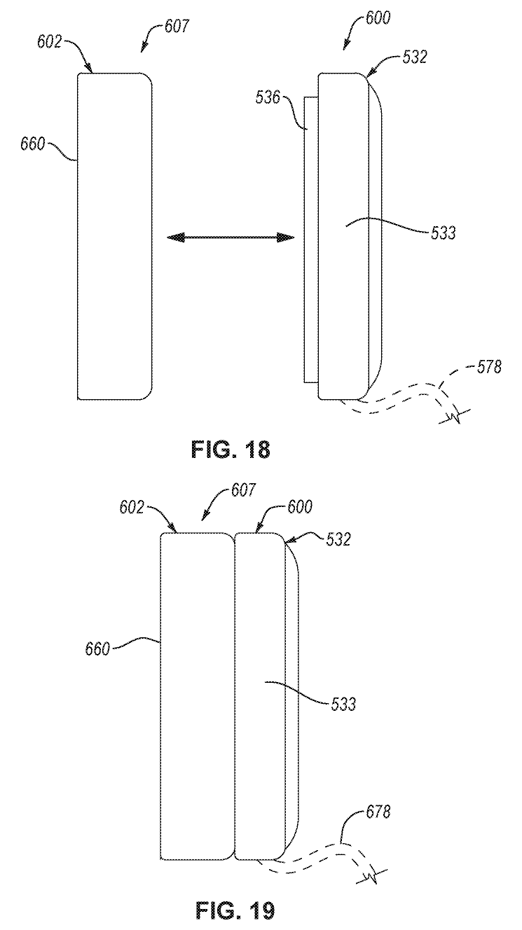

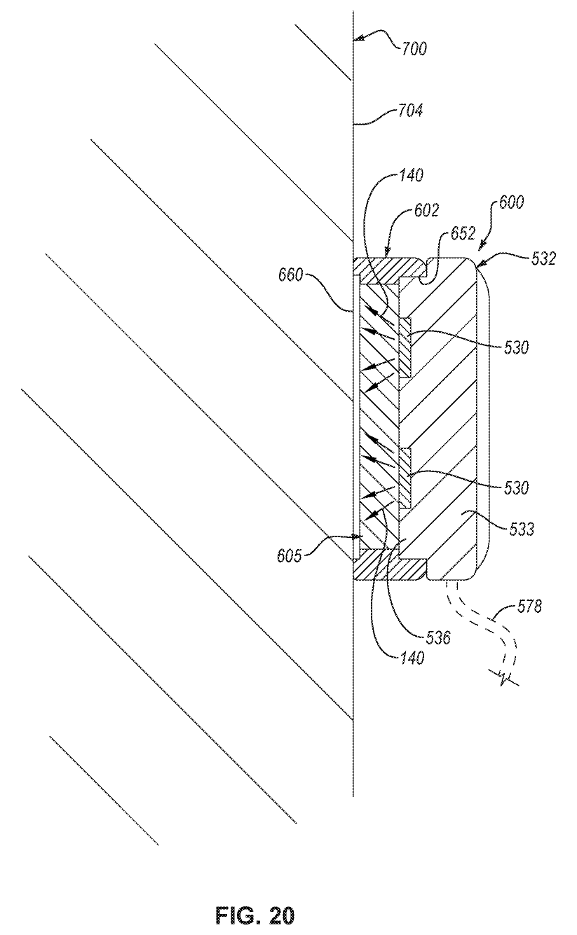

When coupled together, the light emitter 532 and the test attachment 607 form a skin testing device 600, as shown in FIGS. 19 and 20. The test attachment 607 includes some features similar to those of the earpiece 507. For example, the test attachment 607 includes a housing 602 and a light applicator 604. The light applicator 604 is coupled to and at least partially housed within the housing 602. Like the housing 502, the housing 602 is made of a substantially opaque material. The housing 602 also has a ring-like shape or a continuous outer perimeter that defines a through-cavity 654. According to some examples, an entirety of the light applicator 604 is located within the through-cavity 654. In other words, unlike the light applicator 504 of the earpiece 507, no portion of the light applicator 604 of the test attachment 607 extends externally away from the housing 602 (see, e.g., FIGS. 18 and 19). In the illustrated example, the light applicator 604 includes a skin interface surface 660 that faces away from the light emitter 532 when the light emitter 532 and the test attachment 607 are coupled together (see, e.g., FIGS. 15 and 16). The skin interface surface 660 is configured to interface with (e.g., abut) a portion of skin to be tested. Accordingly, the skin interface surface 660 is flat in some examples and slightly contoured in other examples. The skin interface surface 660 can be flush with a distal-most portion of the housing 602, as shown, or slightly recessed relative to the distal-most portion of the housing 602.

Like the housing 502, the housing 602 also includes magnetic coupling elements 650 that are accessible within a receptacle 652 of the test attachment 607. The receptacle 652 is defined between the housing 602 and the light applicator 604 when coupled to the housing 602 (see, e.g., FIG. 16). The magnetic coupling elements 650 are positioned on the housing 602 to correspond with the position of magnetic coupling elements 550 on the light emitter 532

To assemble the skin testing device 600, the test attachment 607 and the light emitter 532 are brought together, as shown in FIG. 18, such that the mating feature 536 of the light emitter 532 is aligned with the receptacle 652 of the test attachment 607. Once aligned, the mating feature 536 can be inserted into the receptacle 652 until the magnetic coupling elements 550 of the light emitter 532 are magnetically coupled to corresponding magnetic coupling elements 650 of the test attachment 607, as shown in FIG. 19.

The skin testing device 600, when assembled, is ready to be placed onto a surface 704 of skin 700 to be tested, as shown in FIG. 20. Proper placement of the skin testing device 600 onto the surface 704 includes placing the skin interface surface 660 in contact with the skin. In this position, the distal-most portion of the housing 602, surrounding the skin interface surface 660, also contacts the surface 704 of the skin. Because the skin of different patients has different photosensitivities, during testing, the light sources 530 of the light emitter 532 are activated to generate incident light 140 at various wavelengths, intensities, and/or durations to help detect baseline light properties associated with inflicting a sun burn on the skin 700, thus detecting a baseline photosensitivity of the skin 700. The incident light 140, being non-coherent, radiates outwardly from the light sources 530 in all directions and passes through the at least partially translucent material of the light applicator 604 before contacting the surface 704 of the skin 700. Some of the incident light 140 passing through the light applicator 604 is absorbed by the housing 602. Because the housing 602 is in contact with the skin 700, all light generated by the light sources 530 either contacts the skin 700 or is absorbed by the housing 602. In this manner, the test attachment 607 prevents generated light from inadvertently escaping the skin testing device 600 when in use during a skin testing procedure.

The ear treatment system further includes a control device that is communicatively coupled with the light emitter 532 and controls operation of the light emitter 532 according to a test mode of operation and a treatment mode of operation. The test mode is only run when the skin testing device 600 (e.g., only a single skin testing device 600) is on a portion of skin to be tested (e.g., skin on an arm, underarm, leg, hand, foot, and the like). In contrast, the treatment mode is only run when the ear treatment device 500 is inserted into an ear, or ear treatment devices 500 are inserted into separate ears, requiring treatment as described above. In one example, the ear treatment system is configured to operate in the test mode when the light emitter 532 is coupled with the test attachment 607 to form the skin testing device 600. The ear treatment system can be quickly configured to operate in the treatment mode by decoupling the test attachment 607 from the light emitter 532, and coupling the light emitter 532 to the earpiece 507. The quick and easy switching between test and treatment configurations using the same system promotes efficiency, cost savings, and simplicity.

In the test mode of operation, the control device is configured to control the light sources 530 to generate light with different characteristics (e.g., different wavelengths, different intensities, and and/or different durations) in a stepwise manner until the skin 700 experiences a requisite degree of erythema (e.g., sun burn). Accordingly, in the test mode, the control device incrementally increases the “exposure” of the light until a detectable skin response, such as erythema or analogous skin condition, is observed, thereby identifying a threshold exposure associated with sunburn onset. In some examples, the control device automates a Minimal Erythema Dose (MED) test. As defined herein, “exposure” may refer to a cumulative light dose associated with any one or more parameters, such as intensity and duration of incident radiation and, in some implementations, wavelength or spectral characteristics of the radiation.

In the treatment mode of operation, the control device is configured to control the light sources 530 to generate light with characteristics based, at least partially, on the threshold exposure determined during the test mode of operation and the condition being treated. In one example, the control device automatically sets an intensity of the generated light based on the threshold exposure. The duration of the application of light to the skin condition of the outer ear canal 202 can be set by the control device as well, or can be set manually or be adjustable by a user. In certain examples, the characteristics of the generated light in the test mode are different than the characteristics of the generated light in the treatment mode.

Control device can be a portable device, such as a laptop, tablet, smartphone, etc., that can be used in a home setting, or a standalone benchtop device that can be used in a clinical setting.

In at least some of the above examples, the earpiece 507 of the ear treatment device 500 and/or the test attachment 607 of the skin testing device 600 can be disposable, such that after a predetermined number of uses (e.g., one), the earpiece 507 and/or the test attachment 607 can be disposed of.

According to certain examples of the ear treatment system disclosed herein, the system can include multiple earpieces 507, each having a differently sized or shaped light applicator 510, and/or multiple test attachments 607, each having a different sized or shaped light applicator 610, to accommodate patients with differently sized physical features.

In the above description, certain terms may be used such as "up," "down," "upper," "lower," "horizontal," "vertical," "left," "right," “over,” “under” and the like. These terms are used, where applicable, to provide some clarity of description when dealing with relative relationships. But, these terms are not intended to imply absolute relationships, positions, and/or orientations. For example, with respect to an object, an "upper" surface can become a "lower" surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.” Moreover, unless otherwise noted, as defined herein a plurality of particular features does not necessarily mean every particular feature of an entire set or class of the particular features.

The term “about” or “substantially” in some embodiments, is defined to mean within +/-5 % of a given value, however in additional embodiments any disclosure of “about” may be further narrowed and claimed to mean within +/- 4 % of a given value, within +/- 3 % of a given value, within +/- 2 % of a given value, within +/- 1 % of a given value, or the exact given value. Further, when at least two values of a variable are disclosed, such disclosure is specifically intended to include the range between the two values regardless of whether they are disclosed with respect to separate embodiments or examples, and specifically intended to include the range of at least the smaller of the two values and/or no more than the larger of the two values. Additionally, when at least three values of a variable are disclosed, such disclosure is specifically intended to include the range between any two of the values regardless of whether they are disclosed with respect to separate embodiments or examples, and specifically intended to include the range of at least the A value and/or no more than the B value, where A may be any of the disclosed values other than the largest disclosed value, and B may be any of the disclosed values other than the smallest disclosed value.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms "first," "second," etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The present subject matter may be embodied in other specific forms without departing from its spirit or essential characteristics. The described examples are to be considered in all respects only as illustrative and not restrictive. All changes which come within the meaning and range of equivalency of the claims are to be embraced within their scope.

What is claimed is:

1. An ear treatment device, comprising:
a light source selectively operable to generate ultraviolet B (UVB) light;
a light disperser coupled with the light source in light receiving communication with the light source; and
a light guide sized to fit within an outer ear canal of an ear and coupled with the light disperser in light receiving communication with the light disperser, wherein the light guide comprises:
an elongate member made of a UVB-transmissive material, comprising a proximal end, fixed to the light disperser, and comprising a distal end, spaced away from the light disperser; and
an optical stop fixed to the distal end of the elongate member and configured to substantially inhibit the transmission of the UVB light longitudinally beyond the distal end of the elongate member.

2. The ear treatment device according to claim 1, wherein the light disperser is made of the UVB-transmissive material.

3. The ear treatment device according to claim 1, wherein:
the optical stop comprises a reflective surface within the elongate member; and
the reflective surface is configured to reflect the light in the elongate member radially outwardly away from the elongate member.

4. The ear treatment device according to claim 3, wherein the optical stop comprises a blunt end outside the elongate member and opposite the reflective surface.

5. The ear treatment device according to claim 3, wherein:
the optical stop comprises a metallic coating; and
the metallic coating defines the reflective surface.

6. The ear treatment device according to claim 1, wherein the UVB light has a wavelength between, and inclusive of, 300 nanometers (nm) and 320 nm.

7. The ear treatment device according to claim 1, further comprising:
a light emitter comprising the light source; and
an earpiece comprising the light disperser and the light guide;
wherein the light emitter is selectively attachable to and detachable from the earpiece.

8. The ear treatment device according to claim 7, wherein:
the light emitter comprises at least one first magnetic coupling element;
the earpiece comprises at least one second magnetic coupling element; and
the light emitter and the earpiece are selectively attachable via magnetic coupling between the at least one first magnetic coupling element of the light emitter and the at least one second magnetic coupling element of the earpiece.

9. The ear treatment device according to claim 1, further comprising a housing surrounding and at least partially enclosing the light disperser, wherein the housing is made of a second UVB-blocking material.

10. The ear treatment device according to claim 1, wherein:
the light disperser has an outer peripheral shape that is non-circular and asymmetrical; and
the light guide is off-center relative to an outer periphery of the light disperser.

11. An ear treatment system, comprising:
two light sources each selectively and independently operable to generate ultraviolet B (UVB) light;
two light applicators each comprising:
a light disperser coupled with a corresponding one of the light sources in light receiving communication with the corresponding one of the light sources; and
a light guide sized to fit within an outer ear canal of an ear and coupled with the light disperser in light receiving communication with the light disperser, wherein the light guide comprises:
an elongate member made of a UVB-transmissive material, comprising a proximal end, fixed to the light disperser, and comprising a distal end, spaced away from the light disperser; and
an optical stop fixed to the distal end of the elongate member and configured to substantially inhibit the transmission of the UVB light longitudinally beyond the distal end of the elongate member; and
a control device communicatively connected with the two light sources via one of wired connection or a wireless connection and configured to independently control operation of each one of the light sources.

12. The ear treatment system of claim 11, further comprising:
two light emitters each comprising a corresponding one of the two light sources; and
two earpieces each comprising a corresponding one of the two light applicators;
wherein each one of the two light emitters is selectively attachable to and detachable from a corresponding one of the two earpieces.

13. The ear treatment system of claim 12, further comprising a test attachment comprising a second light applicator, wherein each one of the two light emitters is separately selectively attachable to and detachable from the test attachment.

14. The ear treatment system of claim 13, further comprising:
two light emitters each comprising a corresponding one of the light sources; and
two earpieces each comprising a corresponding one of the two light applicators;
wherein:
each one of the two light emitters comprises at least one first magnetic coupling element;
each one of the two earpieces comprises at least one second magnetic coupling element;
the test attachment comprises at least one third magnetic coupling element;
each one of the two light emitters and a corresponding one of the two earpieces are selectively attachable via magnetic coupling between the at least one first magnetic coupling element and the at least one second magnetic coupling element; and
the test attachment and each one of the two light emitters are separately selectively attachable via magnetic coupling between the at least one first magnetic coupling element and the at least one third magnetic coupling element.

15. The ear treatment system of claim 13, further comprising:
two light emitters each comprising a corresponding one of the light sources; and
two earpieces each comprising a corresponding one of the two light applicators and a first housing to which the corresponding one of the two light applicators is fixed;
wherein:
the test attachment further comprises a second housing to which the second light applicator is fixed;
for each one of the two light applicators, the light disperser and the light guide extend from and are not surrounded by the first housing; and
the second light applicator is surrounded by the second housing.

16. The ear treatment system of claim 15, wherein:
for each one of the two light applicators, the light disperser continuously converges in a direction away from the first housing and at least a portion of the light guide has a constant diameter along a length of the light guide; and
the second light applicator comprises a flat surface that is flush with a distal-most portion of the second housing.

17. The ear treatment system of claim 13, wherein:
the control device is configured to operate a corresponding one of the two light emitters in a test mode when the corresponding one of the two light emitters is selectively attached to the test attachment; and
the control device is configured to operate the two light emitters in a treatment mode when each one of the two light emitters is selectively attached to the corresponding one of the two earpieces.

18. The ear treatment system of claim 17, wherein:
in the test mode, the control device incrementally increases the exposure of the light generated by the light source of the corresponding one of the two light emitters to determine a threshold exposure of light associated with a detectable skin response; and
in the treatment mode, the control device controls an intensity of the light generated by the light sources of the two light emitters based on the threshold exposure.

19. A method of treating a skin condition of an outer ear canal, the method comprising:
inserting a light guide of an ear treatment device into the outer ear canal of an ear of a patient;
generating light, having a wavelength between, and inclusive of, 300 nanometers (nm) and 320 nm;
passing the light through an elongate member of the light guide in an axial direction along a central axis of the elongate member; and
redirecting the light in the elongate member outwardly away from the central axis, out of the elongate member, and into contact with the skin condition of the outer ear canal.

20. The method of claim 19, further comprising, prior to inserting the light guide of the ear treatment device into the outer ear canal of the ear of the patient:
selectively attaching a test attachment to a light emitter to form a skin testing device;
applying the skin testing device against a portion of skin of the patient away from the ear;
incrementally increasing the exposure of the light until a detectable skin response occurs in the portion of the skin;
identifying the exposure of the light at which the detectable skin response occurs as a threshold exposure;
detaching the test attachment from the light emitter; and
selectively attaching an earpiece to the light emitter, wherein the earpiece comprises the light guide and the earpiece and the light emitter form the ear treatment device.
